(19) United States
(12) Patent Application Publication
(10) Pub. No.: US 2021/0319904 A1
(43) Pub. Date: Oct. 14, 2021

(54) System and Method for Tracking Infectious Disease

(71) Applicant: Medic, Inc., Provo, UT (US)

(72) Inventors: David R. Hall, Provo, UT (US); K. Jeffrey Campbell, Spanish Fork, UT (US)

(73) Assignee: Medic, Inc., Provo, UT (US)

(21) Appl. No.: 17/226,007

(22) Filed: Apr. 8, 2021

Related U.S. Application Data
(60) Provisional application No. 63/007,257, filed on Apr. 8, 2020.

(51) Int. Cl. G16H 50/30; A61B 10/00; A61B 5/01; G16H 40/63; G16H 40/67; G01N 33/487; G01N 33/493; G01N 33/68

(52) U.S. Cl. CPC G16H 50/30; A61B 10/0051; A61B 10/0038; A61B 5/01; G16H 40/63; G01N 2800/26; G01N 33/48792; G01N 33/493; G01N 33/6893; A61B 2562/227; A61B 2562/0271; G16H 40/67

(57) ABSTRACT

A system for tracking an infectious disease in a population comprising an analytical toilet configured to analyte a sample of excreta or sputum from an individual in the population, to thereby determine whether the individual is positive for the infectious disease; a processing module configured to link each positive result with the date and time it was determined; a communication module for communicating data comprising the linked positive result and date and time to an authority, thus providing the authority with information required to track the infectious disease in the population is disclosed.

### CROSS-REFERENCE TO RELATED APPLICATIONS

This application claims priority to U.S. Provisional Patent Application No. 63/007,257 entitled “Method for Obtaining Clearance” filed on 8 Apr. 2020, which is incorporated herein by reference in its entirety.

### TECHNICAL FIELD

The present disclosure relates to a systems and methods for tracking infectious diseases.

### BACKGROUND

The ability to track an individual's health and wellness is currently limited due to the lack of available data related to personal health. Many diagnostic tools are based on examination and testing of excreta, but the high cost of frequent doctor's visits and/or scans make these options available only on a very limited and infrequent basis. Thus, they are not widely available to people interested in tracking their own personal wellbeing.

There can be significant risk to the general public when in close contact with individuals who are infected with a contagious disease, such as a coronavirus. Individuals who may or may not be showing symptoms may be unaware they are infected with a contagious disease and are acting as a disease vector. Closed and confined areas such as airplane cabins, buses, commuter trains, and subway cars are especially robust areas to spread diseases.

### SUMMARY

In a first aspect, the disclosure provides a system for tracking an infectious disease in a population comprising an analytical toilet configured to analyze a sample of excreta or sputum from an individual in the population, to thereby determine whether the individual is positive for the infectious disease; a processing module configured to link each positive result with the date and time it was determined; a communication module for communicating data comprising the linked positive result and date and time to an authority, thus providing the authority with information required to track the infectious disease in the population.

In a second aspect, the disclosure provides a method of tracking an infectious disease in a population comprising providing a toilet configured to analyze a sample of excreta or sputum from an individual in the population, to thereby determine whether the individual is positive for the infectious disease; receiving samples of excreta or sputum from individuals within the population; analyzing each sample; and reporting to an authority each positive result along with the date and time of each positive result.

Further aspects and embodiments are provided in the foregoing drawings, detailed description and claims.

### BRIEF DESCRIPTION OF THE DRAWINGS

The following drawings are provided to illustrate certain embodiments described herein. The drawings are merely illustrative and are not intended to limit the scope of claimed inventions and are not intended to show every potential feature or embodiment of the claimed Inventions. The drawings are not necessarily drawn to scale; in some instances, certain elements of the drawing may be enlarged with respect to other elements of the drawing for purposes of illustration.

FIG. 1 illustrates an exemplary embodiment of an analytical toilet with the lid closed, according to the present disclosure.

FIG. 2 illustrates an exemplary embodiment of an analytical toilet with lid open, according of the present disclosure.

FIG. 3 illustrates an exemplary embodiment of an analytical toilet with lid closed and a portion of the exterior shell removed, according to the present disclosure.

### DETAILED DESCRIPTION

The following description recites various aspects and embodiments of the inventions disclosed herein. No particular embodiment is intended to define the scope of the invention. Rather, the embodiments provide non-limiting examples of various compositions, and methods that are included within the scope of the claimed inventions. The description is to be read from the perspective of one of ordinary skill in the art. Therefore, information that is well known to the ordinarily skilled artisan is not necessarily included.

### Definitions

The following terms and phrases have the meanings indicated below, unless otherwise provided herein. This disclosure may employ other terms and phrases not expressly defined herein. Such other terms and phrases shall have the meanings that they would possess within the context of this disclosure to those of ordinary skill in the art. In some instances, a term or phrase may be defined in the singular or plural. In such instances, it is understood that any term in the singular may include its plural counterpart and vice versa, unless expressly indicated to the contrary.

As used herein, the singular forms “a,” “an,” and “the” include plural referents unless the context clearly dictates otherwise. For example, reference to “a substituent” encompasses a single substituent as well as two or more substituents, and the like.

As used herein, “for example,” “for instance,” “such as,” or “including” are meant to introduce examples that further clarify more general subject matter. Unless otherwise expressly indicated, such examples are provided only as an aid for understanding embodiments illustrated in the present disclosure and are not meant to be limiting in any fashion. Nor do these phrases indicate any kind of preference for the disclosed embodiment.

As used herein, “toilet” is meant to refer to any device or system for receiving human excreta, including urinals.

As used herein, the term “bowl” refers to the portion of a toilet that is designed to receive excreta.

As used herein, the term “base” refers to the portion of the toilet below and around the bowl supporting it.

As used herein, the term “user” refers to any individual who interacts with the toilet and deposits excreta therein.

As used herein, the term “excreta” refers to any substance released from the body including urine, feces, menstrual discharge, saliva, mucus, expectorate, sputum, and anything contained therein or excreted therewith.

As used herein, the term “sputum” refers to a mixture of saliva and mucus coughed up from the respiratory tract, typically as a result of infection or other disease and often examined to aid medical diagnosis.

As used herein, the term “manifold” is intended to have a relatively broad meaning, referring to a device with multiple conduits and valves to controllably distribute fluids, namely water, liquid sample and air.

As used herein, the term “test chamber” is meant to refer broadly to any space adapted to receive a sample for testing, receive any other substances used in a test, and apparatus for conducting a test, including any flow channel for a fluid being tested or used for testing.

As used herein, the term “sensor” is meant to refer to any device for detecting and/or measuring a property of a person or substance regardless of how that property is detected or measured, including the absence of a target molecule or characteristic.

As used herein, the term “microfluidics” is meant to refer to the manipulation of fluids that are contained to small scale, typically sub-millimeter channels. The “micro” used with this term and others in describing this invention is not intended to set a maximum or a minimum size for the channels or volumes.

As used herein, the term “microfluidic chip (MFC)” is meant to refer to is a set of channels, typically less than 1 mm², that are etched, machined, 3D printed, or molded into a microchip. The micro-channels are used to manipulate microfluidic flows into, within, and out of the microfluidic chip.

As used herein, the term “microfluidic chamber” is meant to refer to a test chamber adapted to receive microfluidic flows and/or a test chamber on a microfluidic chip.

As used herein, the term “lab on-chip” is meant to refer to a device that, integrates one or more laboratory functions or tests on a single integrated circuit. Lab-on-a-chip devices are a subset of microelectromechanical systems (MEMS) and are sometimes called “micro total analysis systems” (μTAS).

As used herein, the term “data connection” and similar terms are meant to refer to any wired or wireless means of transmitting analog or digital data and a data connection may refer to a connection within a toilet system or with devices outside the toilet.

As used herein, “biomarker” and “biological marker” are meant to refer to a measurable indicator of some biological state or condition, such as a normal biological processes, pathogenic processes, or pharmacologic responses to a therapeutic intervention. Some biomarkers are related to individual states or conditions. Other biomarkers are related to groups or classifications or states or conditions. For example, a biomarker may be symptomatic of a single disease or of a group of similar diseases that create the same biomarker.

As used herein, “analyte” is meant to refer to a substance whose chemical constituents are being identified and measured.

As used herein, the prefix “nano-” is meant to refer to something in size such that units are often converted to the nano-scale for ease before a value is provided. For example, the dimensions of a molecule may be given in nanometers rather than in meters.

As used herein, “miniaturized electronic system” is meant to refer to an electronic system that uses nanometer scale technology.

As used herein, a “fluidic circuit” is meant to refer to the purposeful control of the flow of a fluid. Often, this is accomplished through physical structures that direct the fluid flow. Sometimes, a fluidic circuit does not include moving parts.

As used herein, a “fluidic chip” is meant to refer to a physical device that houses a fluidic circuit. Often, a fluidic chip facilitates the fluid connection of the fluidic circuit to a body of fluid.

As used herein, a “authority” is meant to refer to a governing body. The governing body can be a government of a nation or governmental agency of a nation. The governing body can preside over a form of public transportation such as an airline, subway, bus, ship, cruise line, city or regional transit or other transportation organization. The governing body can preside over a sports or athletics organization. The governing body may be made up of one or more people. The governing body can establish rules, guidelines or laws that a user must follow in order for a user to use or have access to the services provided.

As used herein, “clearance” is meant to refer to allowance granted to a user who has successfully passed pre-determined requirements. The clearance allows the user to use public transportation, travel across borders, attend amusement parks, attend or participate in sporting events, or in general, participate in activities where large groups of people congregate. The user may also include employees who work for public transportation, amusement parks, sports, border control or other organizations that manage and come into contact with large groups of people. Clearance allows a user to participate in or manage activities that do not affect themselves or others in a negative way. A user who has obtained clearance may also be referred to as one who has been “certified”, “granted permission” or “approved”.

As used herein, “FET” is meant to refer to a field effect transistor, which is a device which uses an electric field to control the current flowing through a device. FETs are also known by the name “unipolar transistor”.

As used herein, a “NAT” is meant to refer to a nucleic-acid test which is a technique used to detect a particular nucleic acid sequence and thus usually to detect and identify a particular species or subspecies of organism, often a virus or bacteria that acts as a pathogen in blood, tissue, urine, etc. NATs differ from other tests in that they detect genetic materials (RNA or DNA) rather than antigens or antibodies. Detection of genetic materials allows an early diagnosis of a disease because the detection of antigens and/or antibodies requires time for them to start appearing in the stool, bloodstream, or other locations.

As used herein, a “NAAT” is meant to refer to a nucleic-acid amplification test to identify small amounts of DNA or RNA in test samples. They can, therefore, be used to identify bacteria, viruses, and other pathogens even when the material of interest is present in very small amounts. NAATs required an additional step to amplify the genetic material by making copies of it. NAATs are typically used in conjunction with such amplification methods as PCR, strand displacement assay (SDA), or transcription mediated assay (TMA).

As used herein, the term “virus” is given its ordinary meaning, namely a small infectious agent, comprised of genetic material within a capsid (protein coat), that replicates only inside the living cells of an organism.

As used herein, a “coronavirus” is a type of virus that causes diseases in birds and mammals. In humans, corona viruses cause respiratory tract infections that, can be mild, such as some cases of the common cold, and others that can be lethal. The coronavirus class of viruses includes alphacoronavirus, betacoronavirus, gammacoronavirus, or deltacoronavirus. More specifically, these viruses include severe acute respiratory syndrome (SARS-CoV), SARS-CoV-2 (also known as COVID-19), and middle east respiratory syndrome (MliRS CoV).

As used herein, the term “port of entry” refers to an entry point into a political jurisdiction including, but not limited to, nations, states, provinces, territories, counties, cities, or the like 3nd any subdivisions thereof.

### Exemplary Embodiments

Toilets present a fertile environment for locating a variety of useful sensors to detect, analyze, and track trends for multiple health conditions. Locating sensors in such a location allows for passive observation and tracking on a regular basis of daily visits without the necessity of visiting a medical clinic for collection of samples and data. Monitoring trends over time of health conditions supports continual wellness monitoring and maintenance rather than waiting for symptoms to appear and become severe enough to motivate a person to seek care. At that point, preventative care may be eliminated as an option leaving only more intrusive and potentially less effective curative treatments. An ounce of prevention is worth a pound of cure.

One particular variety of detection, analysis, and trend tracking is related to biomarkers. “A bio-marker, or biological marker, is a measurable indicator of some biological state or condition. Biomarkers are often measured and evaluated to examine normal biological processes, pathogenic processes, or pharmacologic responses to a therapeutic intervention. Biomarker information can be a valuable resource in providing for the health and wellness of an individual or population. Some of the uses include being used to detect a contagious disease at its earliest stages and monitoring the progression of key health metrics overtime.

The present disclosure relates to a method for detecting and tracking infectious disease. The disclosure further relates to analytical toilets with analytical tools (may also be referred to as a “smart toilet” or a “health and wellness” toilet) which detects, analyzes, and/or tracks the trends of analytes, such as biomarkers, of a user who deposits excreta into the toilet. More specifically, the toilet receives excreta or sputum from a user, processes the excreta or sputum in preparation for analysis, and brings a sample extracted from the excreta or sputum into a testing area for detection of a health condition by a detection system. In some embodiments, sputum deposited upon a probe may also be deposited in the toilet or inserted into a receptacle for analysis. The health condition may include, for example, if the user has alcohol or illicit drugs in their body, high blood pressure, fever, diabetes or congestive heart failure. In an exemplary embodiment, the detection system in an analytical toilet described herein may be able to detect one or more viruses or bacteria, in particular, the detection system can detect contagious viruses such as from the coronavirus class including alphacoronoavirus, betacoronovirus, gammacoronavirus, or deltacoronavirus. More preferably, the viruses that may be detected include SARS-CoV-2 (also known as COV1D-19) or SARS-CoV. Other contagious viruses include influenza A (e.g., H1N1, H1N2), dengue, bacterial meningitis, viral meningitis, Ebola and Zika. The detection area may comprise a NAT, NAAT, or FET-based detection system. After the toilet has finished with the sample, the toilet purges the sample from the toilet in preparation for receiving a new sample of excreta or sputum. The information received from the detection system in the analytical toilet may be provided to the user and to health authorities. The information provided to authorities may be aggregated from multiple users and the results may or may not be connected to individual users.

In various exemplary embodiments, the analytical toilet comprises test devices that are designed to perform one or more of a variety of laboratory tests. Any test, that could be performed in a medical or laboratory setting may be implemented in an analytical test device. These tests may include measuring pulse, blood pressure, blood oxygenation, electrocardiography, body temperature, body weight, excreta or sputum content, excreta or sputum weight, excreta or sputum volume, excreta or sputum temperature, excreta or sputum density, excreta flow rate, and other health and wellness indicators. This information may also be used to grant or not grant clearance to a user.

In accordance with the present disclosure, an analytical toilet that includes an infrastructure for multiple health and wellness analysis tools is provided. This provides a platform for the development of new analytical tools by interested scientists and companies. Newly developed tests and diagnostic tools may be readily adapted for use in a system having a consistent, tool interface.

In various exemplary embodiments, the analytical toilet provides a fluid processing manifold that collects and routes samples from the toilet bowl to various scientific test devices and waste handling portals throughout the device.

In various exemplary embodiments, the analytical toilet provides multiple fluid sources via a manifold system. The manifold is adapted to connect to a plurality of analytic test devices adapted to receive fluids from the manifold. The manifold is designed to selectively provide a variety of different fluid flows to the analytical test device. These fluids may include, among others, excreta samples, sputum, buffer solutions, reagents, water, cleaners, biomarkers, dilution solutions, calibration solutions, and gases such as air or nitrogen. These fluids may be provided at different pressures and temperatures. The manifold and analytical test device are also adapted to include a fluid drain from the analytical test devices.

In various exemplary embodiments, the manifold system provides a standardized interface for analytical test devices to connect and receive ail common supplies (e.g., excreta samples, sputum, flush water), data, and power. Common supplies may be supplied from within (e.g., reagents, cleaners) or without (e.g., water) the toilet system. The analytical test devices may be designed to receive some or all of the standardized flows. The analytical test devices may also include storage cells for their own unique supplies (e.g., test reagent).

In various exemplary embodiments, the manifold is adapted to direct fluids from one or more sources to one or more analytical test devices. The manifold and analytical test devices are designed such that analytical test devices can be attached to and detached from the manifold making them interchangeable based on the needs of the user. Different analytical test devices are designed to utilize different, test methods and to test excreta or sputum samples for different constituents, such as contagious bacteria or viruses.

In various exemplary embodiments, the analytical toilet, provides an electrical power connection and a data connection for the analytical test device, in a preferred embodiment, the electrical power and data connections use the same circuit. In various exemplary embodiments, the toilet is provided with pneumatic and/or hydraulic power to accommodate the analytical test devices.

In various exemplary embodiments, the analytical toilet platform performs various functions necessary to prepare samples for examination. These functions include, but are not limited to, separating urine from feces, diluting or concentrating samples, large particle filtration, sample agitation, and adding reagents.

In various exemplary embodiments, the analytical toilet also provides, among other things, fluid transport, fluid metering, fluid valving, fluid mixing, separation, amplification, storage and release, and incubation. The analytical toilet also is equipped to provide cleansers, sanitizers, rinsing, and flushing of all parts of the system to prevent cross-contamination of samples. In some embodiments, the system produces electrolyzed water for cleaning.

In various exemplary embodiments, one layer of the fluidic manifold is dedicated to macro-scale mixing of fluids. Sample, diluents, and reagents are available as inputs to the mixers. The mixing chamber is placed in series with all other scientific test, devices, allowing bulk mixed sample to be routed to anywhere from one to all stations (i.e., analytical test device interfaces) for analysis. Mixing may also occur in an analytical test device.

In various exemplary embodiments, urine samples are filtered for large particulates at. the fluid ingress ports of the manifold. The fluid manifold uses a network of channels along with simple valves to route prepared stool samples to one of several scientific test devices located on the platform.

In various exemplary embodiments, the manifold is constructed using additive layers, and different layers can be customized for particular applications. Standard ports and layouts are used for interfacing with external components, such as pressure sources and flow sensors. In general, characteristic channel volumes at the bottom of the manifold stack are on the order of milliliters. At the top of the manifold stack is the microfluidic science device, which will interface simultaneously with multiple microfluidic. chips using standardized layout and pressure seals.

In various exemplary embodiments, the system is adapted to work with a variety of actuation technologies that may be used in the analytical test devices. The system provides electronic and fluidic interconnects for various actuator technologies and supports OEM equipment. In a preferred embodiment, the system is adapted to work with actuator modules that can be attached to the sample delivery manifold and controlled by a central processor. The system platform supports an inlet and outlet for the pressure transducer that interfaces with the fluidic manifold, and electronic or pneumatic connections where required. The system supports a variety of macro- and microfiuidic actuation technologies including, but not limited to, pneumatic driven, mechanical pumps (e.g., peristaltic), on-chip check-valve actuators (e.g., piezo-driven or magnetic), electroosmotic driven flow, vacuum pumps, and capillary or gravity driven flow (i.e., with open channels and vents).

One benefit, of the present disclosure is the detection, monitoring, and tracking of a user's biomarkers without having any inconvenience aside from what they would otherwise do using the toilet. Without the present disclosure, among other things, people often have to manually collect samples of excreta, use equipment they are less familiar with than a toilet, or wait longer for analysis and results. Each of these things can negatively impact a user's experience and/or the quality or accuracy of the results. Additionally, the present disclosure describes tracking the trend data of a population of users. This may be beneficial in the detection of an emergence of an epidemic or even a pandemic.

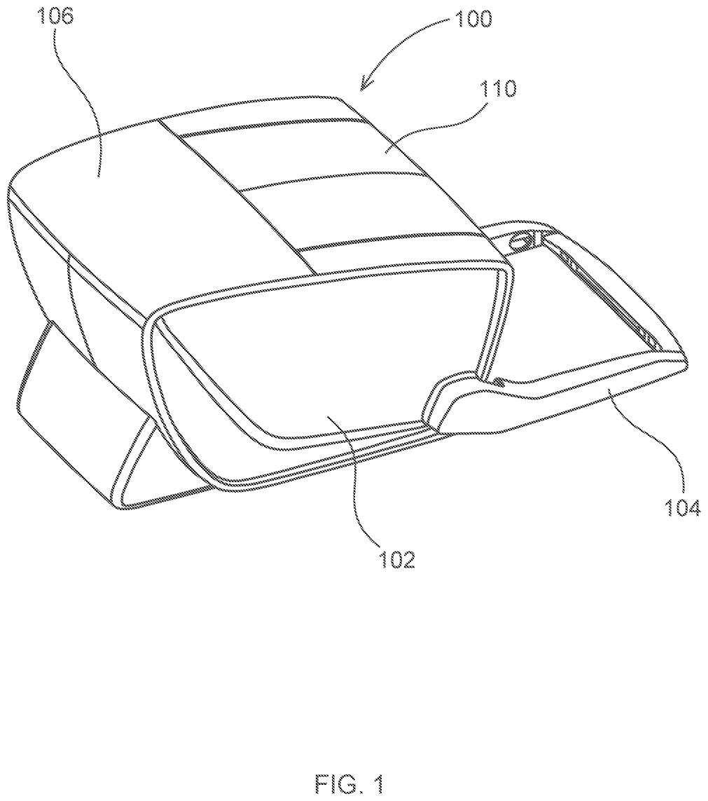

Now referring to FIGS. 1-3, a preferred embodiment of an analytical toilet 100 is shown. FIG. 1 illustrates the analytical toilet 100 with the lid 110 closed, according to an embodiment of the disclosure. FIG. 1 further shows exterior shell 102, foot, platform 104 and rear cover 106. The lid 110 is closed to prevent a user from depositing urine in toilet 100 until the toilet is ready for use.

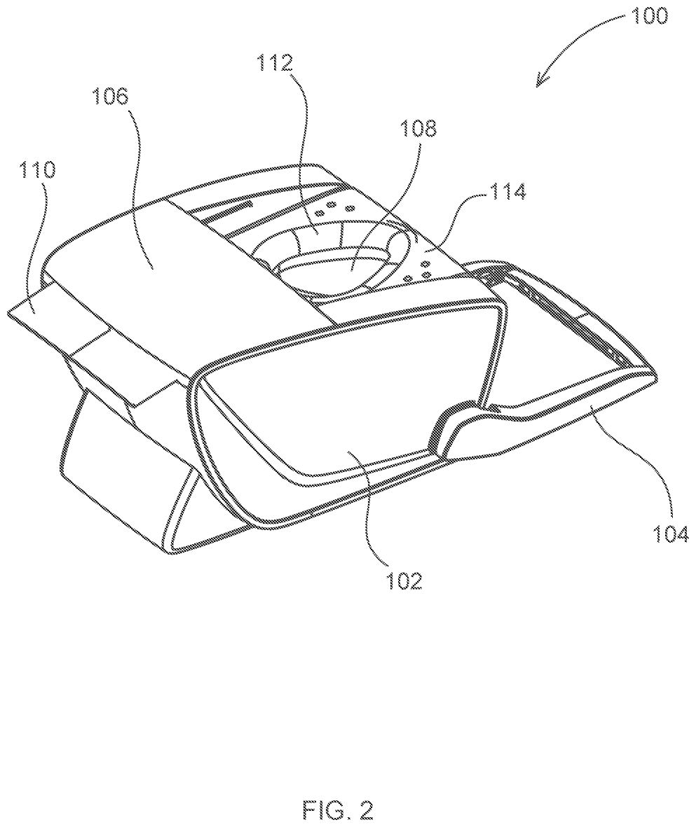

FIG. 2 illustrates toilet 100 with lid 110 open, according to an embodiment of the disclosure. Toilet 100 includes exterior shell 102, rear cover 106, bowl 108, seat 112, lid 110, fluid containers 114 and foot platform 104 Housed within toilet 100 are a variety of features, including equipment, that facilitate receiving excreta, processing urine for analysis, analyzing urine, and disposing of urine. FIG. 2 shows toilet 100 with lid 110 open so a user can sit on seat 112 and deposit urine in toilet 100.

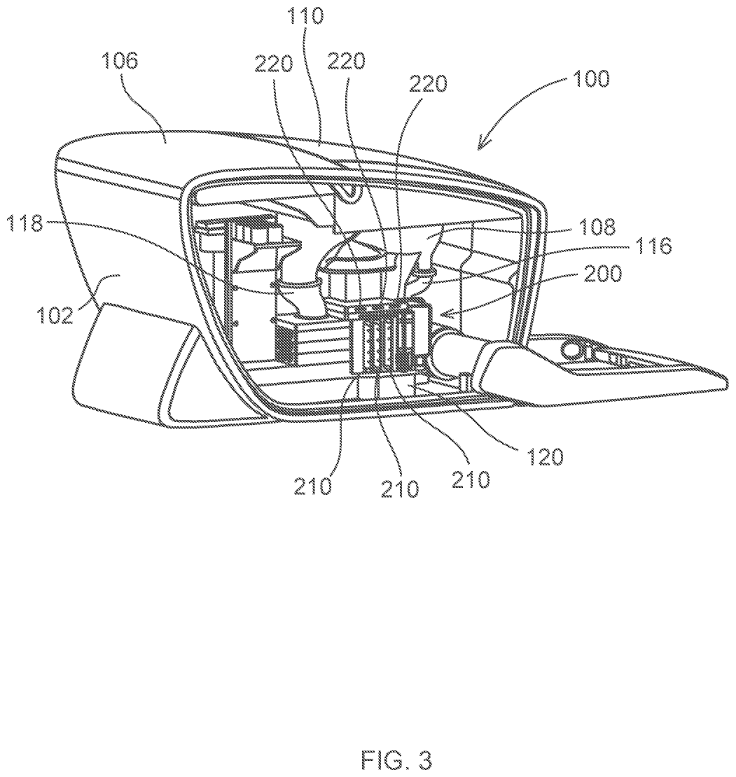

FIG. 3 illustrates toilet 100 with lid 110 closed and a portion of exterior shell 102 removed, according to an embodiment of the disclosure. This allows access to equipment housed within toilet 100. With exterior shell 102 removed, base 120, urine collection pipe 116, feces collection pipe 118, and manifold area 200 is visible. Urine collection pipe 116 further comprises a passageway to deliver a urine sample to the manifold area 200 and to a detection system. Manifold area 200 includes test areas 210 and fluidic chip slots 220. Preparation and/or analysis of sample can selectively t3ke place in a test area 210 or fluidic chip slot 220. Manifold area 200 is the area where analysis takes place. A filter may be added over the entrance of the urine collection pipe 116 to prevent solid material, such as feces or toilet paper, from entering the pipe.

A manifold 200 is located below the bowl 108. The manifold 200 comprises a plurality of fluid paths. These fluid paths allow the manifold 200 to move fluids between the bowl 108, fluid containers 114, outside sources (e.g., municipal water supplies), other sources (e.g., air or water electrolyzing unit), analytical test devices 210, and the toilet outlet. The analytical test devices 210 make up a detection system for one or more viruses. The manifold 200 also provides electrical power and data connections to the analytical test devices 210. The manifold 200 can also directly pass fluids and/or solids from the bowl 108 to the toilet outlet. The manifold 200 is adapted to provide receptacles 210 with standardized connection interfaces for multiple analytical test devices 210. The manifold 200 is shown here with multiple fluid sources 201 for the analytical test device 210. In various embodiments, the manifold 200 may include receptacles for more than one type of analytical test device 210 (e.g., different sizes, fluid supply needs, etc.). In various exemplary embodiments, the analytical test device 210 includes multiple inlets in fluid communication with the manifold 200. The analytic test device 210 may also include at least one outlet or drain in fluid communication with the manifold 200

In various exemplary embodiments, manifold may comprise a microfluidic system to isolate and transport a sample, add, and mix reagents if appropriate, filter out solids, and test the sample for one or more coronaviruses on a small scale (i.e., sub-millimeter scale) in a health and wellness analytical toilet described herein. The microfluidic system may comprise an open microfluidic system, continuous-flow microfluidic system, droplet-based microfluidic system, digital microfluidic system, nanofluidic system, paper-based microfluidic system or combinations thereof.

A microfluidic.-based contagious disease detection system may be located on a microfluidic chip (MFC). In a preferred embodiment, the MFC includes a test chamber with a lab-on-chip (“LoC”) (also known as “test-on-chip”). The LoC may be designed to perform one or more laboratory tests. In various exemplary embodiments, one or more microfluidic chips (MFCs) may be removed or added to the toilet system as desired or needed at any given time, such as for different biomarker tests. In exemplary embodiments, a DNA microfluidic chip may be used as a component in a virus biomarker sensor in a health and wellness analytical toilet. The DNA chip may comprise a DMA microarray, such as the GeneChip DNAarray (Affymetrix, Santa Clara, Calif., USA). The DNA microarray comprises one or more pieces of DNA (probes) for virus biomarker detection. The MFC may comprise one or more affixed proteins in an array-like fashion. In an exemplary embodiment, the proteins are monoclonal antibodies for detection of antigens. A DNA microarray may also be used to detect RNA-based virus genetic material.

In various exemplary embodiments, a MFC is designed to use very small quantities of reagent. In various exemplary embodiments, reagents are dispensed using technology similar to that used in inkjet printers to dispense ink. In some embodiments, an electrical current is applied piezoelectric crystal causing its shape or size to change forcing a droplet of reagent to be ejected through a nozzle. In some embodiments, an electrical current is applied to a heating element (i.e., resistor) causing reagent to be heated into a tiny gas bubble increasing pressure in the reagent vessel forcing a droplet of reagent to be ejected.

In various exemplary embodiments, the toilet fluidic manifold provides routing. Interconnecting levels of channels allows routing from one port to all others. Each channel includes an accumulator; allows for constant pressure pumping of all active channels simultaneously, while time-multiplexing pump-driven inflow.

In various exemplary embodiments, the manifold has reaction chambers built in for general purpose mixing and filtering operations. Each chamber has a macro-sized channel through which the manifold delivers a sample extracted from urine (filling the reaction chamber), and the chamber has a micro -sized channel. Pumps located internal or external to the manifold drive fluid into the reaction chamber, and into the micro-sized channel. A valve at the output of the macro-channel, and possibly at the output of the micro-channel, controls fluid direction as it exits the reaction chamber.

Microfluidic applications require support infrastructure for sample preparation, sample delivery, consumable storage, consumable delivery or replenishment, and waste extraction. In various exemplary embodiments, the manifold includes integrated support for differential pressure applications, pneumatic operations, sample and additive reservoirs, sample accumulators, external pumps, pneumatic pressure sources, active pump pressure (e.g., peristaltic, check-valve actuators, electro-osmotic, electrophoretic), acoustic or vibrational energy, and light-interaction (e.g., spectrometer, laser, UV, magnification). The acoustic energy source may be a high frequency (54 MHz) bulk acoustic wave (BAW) actuator.

In various exemplary embodiments, the manifold interface has a matrix of ports, possibly laid out in a regular grid. These ports may be activated or dosed via an external support manifold. Routing is fully programmable.

In various exemplary embodiments, the manifold directs one or more fluids to the analytical test device or an MFC analytical test device to cleanse the devices. These may include cleaning solutions, disinfectants, and flushing fluids, in various exemplary embodiments, the manifold directs hot water or steam to clean sample, reagents, etc. from the devices. In various exemplary embodiments, the toilet systems using oxygenated water, ozonated water, electrolyzed water, which may be generated on an as-needed basis by the toilet system (this may be internal or external to the toilet).

In some embodiments, the manifold comprises a heater. The heater may be used to drive off water and concentrate a urine sample.

In various exemplary embodiments, waste from the MFCs is managed based on its characteristics and associated legal requirements. Waste that can be safely disposed is discharged into the sewer line. Waste that can be rendered chemically inert (e.g., heat treatment, vaporization, neutralization) is processed and discharged. Waste that cannot be discharged or treated in the toilet system is stored, and sequestered if necessary, for removal and appropriate handling.

In various exemplary embodiments, the manifold creates sequestered zones for each of these waste categories and ensures that all products are properly handled. In various exemplary embodiments, the manifold directs flushing water and/or cleansing fluids to clean the manifold and MFC. In some embodiments, high-pressure fluids are used for cleaning. In such an embodiment, the high-pressure fluids are not used in the MFC. In some embodiments, the MFC is removed from the backplate interface and all ports are part of the high-pressure cleansing and/or rinse.

There are many ways to incorporate detection systems into the toilet, the selection of which will depend on various factors, including ease of manufacture and maintenance, target market, physical constraints, frequency of use compared to other desired functions of the toilet, and cost. In one preferred embodiment, the detection system is integrated with a fluidic circuit. More preferably, the fluidic circuit is on a fluidic card. Still more preferably, the fluidic circuit on the fluidic card is a microfiuidic circuit on a micro fluidic card. Yet more preferably, the microfiuidic circuit interfaces with n3no-scale fluidic circuits. Preferably, the fluidic card is inserted into a slot or receptacle of the toilet which connects the fluid circuit on the card to the toilet's fluidic delivery system, enabling the card to receive the sample derived from excreta or sputum. Alternatively, the detection system is part of a larger device that, may be attached to the toilet, such as a device that processes and/or analyzes the sample extracted from excreta or sputum. Alternatively, the detection system is built into the toilet rather than being on a card. Alternatively, the sensor is external to the remainder of the toilet and is connected to receive and/or return fluid from the toilet, such as may be accomplished by connecting the sensor to part of the toilet with tubes or pipes.

In some embodiments, analytical toilet may further comprise one or more sensors to collect information such as the temperature, blood pressure, pulse, body weight and electrocardiography (EKG), blood oxygenation, water retention, skin moisture, excreta content, excreta weight, excreta volume, excreta temperature, excreta density, and excreta flow rate of the user. The sensors may be located in the seat, lid, or other location in the toilet. To gain an accurate body temperature of a user, an infra-red (IFt) camera may be located in the bowl such that the temperature of a stream of urine may be taken as it is leaving the body of the user. This would provide an accurate method of determining the core temperature of the user, in a preferred embodiment, the seat Is attached to the toilet via a powered quick disconnect system that allows the seat to be interchangeable. This facilitates installing custom seats to include user-specific tests based on known health conditions. It also facilitates installing upgraded seats as sensor technology improves.

The toilet may be located in a transportation terminal such as an airport terminal, bus station, train station, ferry station, cruise line terminal, or taxi station. The toilet may be located at home, a nursing home, doctor's office, hospital, gymnasium, or fitness center. In some embodiments, the toilet may be located in a privately owned and exclusive facility such as a lounge or a spa. The lounge or spa may provide a more comfortable, private and sterile setting to utilize the toilet. The lounge or spa may be sex or gender specific. The lounge or spa may grant memberships and require a fee to use the lounge service. For example, a monthly or yearly fee may be charged.

The user identification unit may be used to enable the smart features of the toilet, and Identification is necessary to connect the data to a user profile. However, in a preferred embodiment, the analytical toilets may be used and accessed like normal washroom facilities by anyone who chooses not to utilize the other features.

In various exemplary embodiments, the toilet system passively monitors usage to test users without identifying individuals or connecting the data obtained to an individual. This data may be used to track trends among users, identify new outbreaks (e.g., monitoring for the appearance of seasonal flu), or group monitoring among other purposes. In some such embodiments, a sample that indicates the presence of 3 contagious pathogen (e.g., CoViD-19 virus) may be tracked back to an individual or small group of users using passive monitoring and identification such as facial recognition applied to recordings of users entering and/or exiting the toilet system and biometric data such as weight, gender, height, etc. Persons who might have provided the sample may then be tested to determine if any are infected. Such an embodiment may be placed in a public location (e.g., airport, bus or train terminal, public building) or a location with limited access (e.g., private building, airplane, train).

In various exemplary embodiments, the system includes a processing module that records the results from each test along with the date, time, and location of the test. Other biometric data (e.g., gender, height, weight, body temperature, blood pressure) regarding the user may also be included. A communication module transmits the data from the analytical toilet to an authority (e.g., health agency) to track the occurrence of positive tests. Some data from negative tests may also be recorded and transmitted.

A user may next leave a sample of excreta or sputum in an analytical toilet. The analytical toilet processes and prepares the excreta or sputum sample. The sample may then be transferred using a passage to a detection system. One or more detection systems may be installed in the analytical toilet such as a DNA microarray, NAT, NAAT or FFT-based detection system. The detection system analyzes the sample and other health and wellness data taken by the toilet and determines if the user is infected. This includes being infected with a contagious disease such as coronavirus, dengue, bacterial or viral meningitis, Zika, Ebola, or influenza A. This may also include testing for temperature, blood pressure, pulse, body weight, electrocardiography (EKG), blood oxygenation, water retention, skin moisture, excreta content, excreta weight, excreta volume, excreta temperature, excreta density, and excreta flow rate of the user. In some instances, the user may not have an infection but may have congestive heart failure, high or low blood pressure, or another health condition. In this instance, the user may be advised to consult a medical professional.

The analytical toilet processes the computer readable data from the detection system and sends the information to the user, in this instance, the information could not be sent to an authority as identification of the user was not set up before the test. This prevents fraudulent activity from testing and analysis of one user being sent in place of another user. This method allows for a user to be tested for their own personal and confidential information. The user may choose to keep the information private and be re-tested using an analytical toilet at a later date when the user may be cured of a disease, healed of any condition, or no longer under the influence of alcohol or an illicit drug. The user may have it sent to their health care provider.

In another embodiment, a user may link to the analytical toilet system to substantiate their identity, leave a sample of excreta or sputum to be tested, then have the option of keeping the information private or sending it directly to an authority for clearance. The results may or not be permanently linked to the user.

When the user uses the analytical toilet, a time stamp may be associated with the health and wellness information collected by the toilet. This is especially important for control of the spreading of contagious diseases amongst the general public. In any of the methods described to transfer health and wellness data from an analytical toilet, the data may be uploaded to a cloud storage center. The data may be retrieved from the cloud by the user, an authorized entity, or a health care provider.

Following use of the analytical toilet, the toilet may prepare the system for future analysis by removing from the test area waste products and other things that might contaminate the next analysis. This could include flushing the detection system, adding a buffer or stabilizing solution, or adding a gas to remove ail liquid from the system. There are various options to clean, sanitize, and/or prepare the various components of the detection system between uses of the toilet. In one preferred embodiment, hot water is run through the fluidic circuits. In another preferred embodiment, oxygenated water is run through the fluidic circuits, in yet another preferred embodiment, a gas is run through the fluidic circuits to remove any liquid from being in contact with the detection system. Alternatively, cleaning and/or preservation agents are run through the fluid circuits.

In various exemplary embodiments, the lid may contain health and wellness sensors that interact with the user's back or that analyze gases in the bowl after the lid is closed.

In various exemplary embodiments, the analytical toilet includes software and hardware controls that are pre-set so that any manufacturer can configure their devices (i.e., analytical test devices) to work in the system. In a preferred embodiment, the system includes a software stack that allows for data channels to transfer data from the sensors in the medical toilet to cloud data systems. The software and hardware controls and/or software stack may be stored in the analytical toilet or remotely. This would allow scientists to place sensors, reagents, etc. in the system to obtain data for their research. It also allows user data to be individually processed, analyzed, and delivered to the user, or their health care provider, digitally (e.g., on a phone, tablet, or computer application). The seat may also contain sensors to measure fluid levels in the toilet. This could include proximity sensors. Alternatively, tubes in fluid communication with the bowl water could be used to determine changes to bowl fluids (e.g., volume, temperature, rate of changes, etc.).

The toilet disclosed herein has many possible uses, including private and public use. Whether for use by one individual, a small group of known users, or general public use, the toilet can detect, monitor, and create one-time and/or trend data for a variety of analytes, such as virus biomarkers. This data can be used to prompt a user to seek additional medical, health, or wellness advice or treatment; track or monitor a user or population's known condition; and provide early detection or anticipation of a disease or another condition of which a user or population may wish to be aware. The analytical toilet described herein may be used for travelers seeking to pass through a continental, national, state, city, county, regional, or other border. Mobile units may be developed for a population under quarantine.

While the present disclosure often notes the detection system and other components supporting excreta and sputum analysis are located within the toilet, it is possible that some or ail of the components are located outside of the toilet. For example, the sample preparation, detection, and processing equipment may be a separate unit adjacent to the toilet, which cooperates with the toilet to automatically or semi-automatically receive excreta or sputum, prepare a sample of excreta or sputum for analysis, test the sample, discard the sample, and prevent cross contamination by cleaning and/or sterilizing portions of the toilet and external equipment that do any portion of the described process.

All patents, published patent applications, 3nd other publications referred to herein are incorporated herein by reference. The invention has been described with reference to various specific and preferred embodiments and techniques. Nevertheless, it is understood that many variations and modifications may be made while remaining within the spirit and scope of the invention.

What, is claimed is:

1. A system for tracking an infectious disease in a population comprising:
an analytical toilet configured to analyze a sample of excreta or sputum from an individual in the population, to thereby determine whether the individual is positive for the infectious disease;
a processing module configured to link each positive result with the date and time it was determined;
a communication module for communicating data comprising the linked positive result and date and time to an authority, thus providing the authority with information required to track the infectious disease in the population.

2. The system of claim 1, wherein the toilet is configured to detect the presence of one or more pathogens or fragments of one or more pathogens in the excreta or sputum.

3. The system of claim 2, wherein the toilet comprises at least one sensor configured to detect nucleic acids or proteins indicative of the presence of the one or more pathogens or the fragments of the one or more pathogens in the excreta or sputum.

4. The system of claim 3, wherein the at least one sensor utilizes a field effect transistor.

5. The system of claim 1, further comprising a thermal sensor for detecting body temperature and wherein the processor is further configured to the link body temperature with each positive result.

6. The system of claim 1, wherein the system comprises a plurality of like toilets deployed at a port of entry and the population comprises individuals crossing a border.

7. The system of claim 1, wherein the system comprises a plurality of like toilets deployed at a transportation hub and the population comprises individuals beginning, continuing, or ending a trip.

8. The system of claim 1, wherein the system comprises a plurality of like toilets deployed at multiple sites, wherein the data communicated includes the location of each positive result, and wherein the system further comprises a central data processing module configured to receive communicated data and provide trend information to the authority.

9. The system of claim 1, wherein the system alerts an authority to a test result indicating the presence of a contagious disease along with any other data collected on the infected user.

10. The system of claim 9, wherein the alert includes the gender, height, and weight of the user.

11. A method of tracking an infectious disease in a population comprising:
providing a toilet configured to analyze a sample of excreta or sputum from an individual in the population, to thereby determine whether the individual is positive for the infectious disease;
receiving samples of excreta or sputum from individuals within the population;
analyzing each sample; and
reporting to an authority each positive result along with the date and time of each positive result.

12. The method of claim 11, wherein the toilet is configured to detect the presence of one or more pathogens or fragments of one or more pathogens in the excret3 or sputum.

13. The method of claim 11, wherein the toilet comprises at least one sensor configured to detect nucleic acids or proteins indicative of the presence of the one or more pathogens or the fragments of the one or more pathogens in the excreta or sputum.

14. The method of claim 11, further comprising using a thermal sensor to detect body temperature and linking link body temperature with each positive result.

15. The method of claim 11, wherein the method comprises deploying a plurality of like toilets at a port of entry, to thereby track the infectious disease within the population comprising individuals crossing a border.

16. The method of claim 11, wherein the method comprises deploying a plurality of like toilets at a transportation hub, to thereby track the infectious disease within the population comprising individuals beginning, continuing or ending a trip.

17. The method of claim 11, wherein the method comprises deploying a plurality of like toilets at multiple sites, and further comprises reporting the location of each positive result.

18. The method of claim 11, wherein data on positive results is tabulated by the authority to generate trend information relating to the infectious disease.

19. The method of claim 11, wherein an alert is provided to an authority when a test result indicates the presence of a contagious disease along with any other data collected on the infected user.

20. The method of claim 19, wherein the alert includes the gender, height, and weight of the user.
